(19) United States
(12) Patent Application Publication
(10) Pub. No.: US 2025/0189503 A1
(43) Pub. Date: Jun. 12, 2025

(54) Methods and Apparatus to Detect Allergens Using a Lateral Flow Test

(71) Applicants: Ariana E. Aghevli, Highlands Ranch, CO (US); Andrew J. Frels, West Lafayette, IN (US); Allison C. Lao, Macomb, MI (US); Aavya N. Srivastava, Princeton, NJ (US)

(72) Inventors: Ariana E. Aghevli, Highlands Ranch, CO (US); Andrew J. Frels, West Lafayette, IN (US); Allison C. Lao, Macomb, MI (US); Aavya N. Srivastava, Princeton, NJ (US)

(21) Appl. No.: 18/976,872

(22) Filed: Dec. 11, 2024

Related U.S. Application Data
(60) Provisional application No. 63/608,672, filed on Dec. 11, 2023.

(51) Int. Cl. G01N 33/02; G01N 1/28

(52) U.S. Cl. CPC G01N 33/02; G01N 1/286; G01N 2001/2866

(57) ABSTRACT

Methods and apparatus relating to detection of allergens using a lateral flow test are described. In an embodiment, an apparatus comprises: a food processor to receive a food sample and a lid to engage the food processor mechanically to grind the food sample. A mixture of a lateral flow test liquid and the ground food sample saturates a portion of a lateral flow test material to indicate whether the food sample includes an allergen. Other embodiments are also disclosed and claimed.

### CROSS-REFERENCE TO RELATED APPLICATIONS

The present application claims priority from and is a non-provisional application of U.S. Provisional Application No. 63/608,672, filed Dec. 11, 2023, entitled “METHOD AND APPARATUS TO DETECT ALLERGENS USING A LATERAL FLOW TEST,” which is hereby incorporated herein and for all purposes.

### FIELD

The present invention relates generally to medical devices and, more particularly, to techniques for detecting allergens using a lateral flow test.

### BACKGROUND OF THE INVENTION

With the rise of allergies, it has become imperative for at least some people to detect allergens in food prior to consumption or exposure. While different food providers may attempt to provide allergen free choices, the risk of contamination (whether direct or indirect) can pose a severe reaction in some users.

### BRIEF DESCRIPTION OF DRAWINGS

The detailed description is provided with reference to the accompanying figures. In the figures, the left-most digit(s) of a reference number identifies the figure in which the reference number first appears. The use of the same reference numbers in different figures indicates similar or identical items.

FIG. 1 illustrates a block diagram of a system to detect allergens using a lateral flow test, according to an embodiment.

FIG. 2 illustrates various views of a food sample processor, according to an embodiment.

FIG. 3 illustrates various views of a lid, according to an embodiment.

FIG. 4 illustrates various views of a pull tab, according to an embodiment.

FIG. 5 illustrates the various portions of an allergen detection device, according to an embodiment.

### DETAILED DESCRIPTION OF THE INVENTION

In the following description, numerous specific details are set forth in order to provide a thorough understanding of various embodiments. However, various embodiments may be practiced without the specific details. In other instances, well-known methods, procedures, and components have not been described in detail so as not to obscure the particular embodiments.

As discussed above, the risk of contamination (whether direct or indirect) can pose a severe reaction in some users. Moreover, some current devices capable of detecting gluten require multiple components and/or fail to provide a method to process food samples into a testable product. These solutions generally require the user to break up the food sample themselves, e.g., by using a blender or fork and knife. Unfortunately, these limitations hinder detection of gluten readily and may cause issues for at least some users with a gluten allergy. Accordingly, users currently do not have a good way to test if their food contains gluten and other allergens on-the-go.

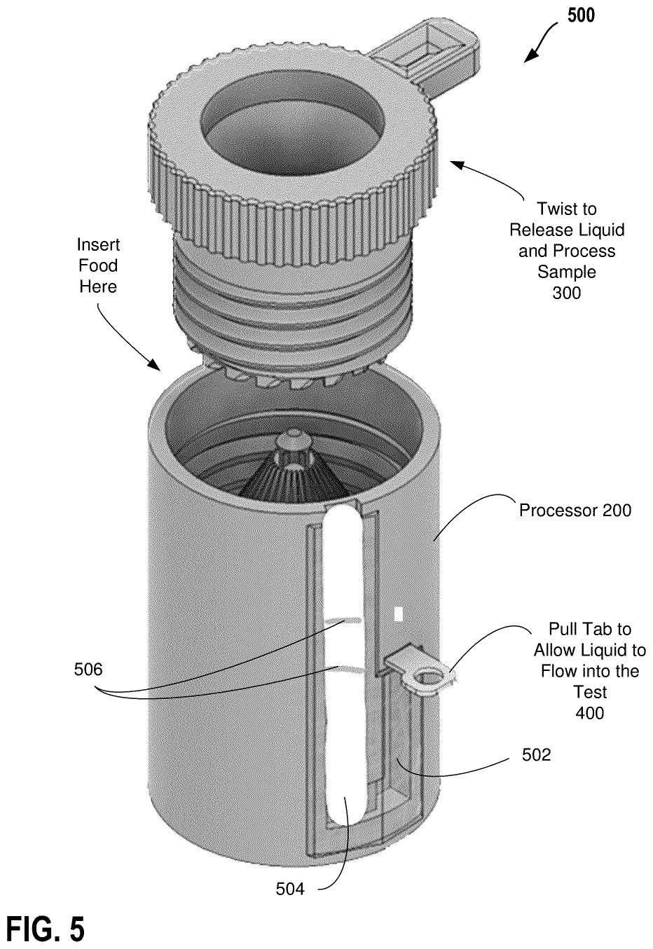

To this end, some embodiments provide techniques to detect allergen(s) (such as gluten, peanut, tree nut, soy, shell fish, etc.) using a lateral flow test. In some embodiments, the apparatus used (e.g., shown in FIG. 5) may be a one-time use apparatus to ensure no cross contamination between different samples. In at least one embodiment, the apparatus is capable of testing for different allergens.

In an embodiment, a method processes the food sample without the use of any extraneous or separate equipment. By eliminating the need to process the food separately, users no longer have to come in contact with the food sample (which in part avoids or limits potential exposure to an allergen that may be present in the food sample). This approach also enables the users to test a wide range of food types, including but not limited to solids and liquids.

In one embodiment, a device includes the grinding mechanism which is integrated with a lateral flow test in order to make the test self-contained. This differs from other products currently on the market, like the NIMA™ sensor and GlutenTox® Home, because both require the food sample to be first processed and that multiple, separate components be used.

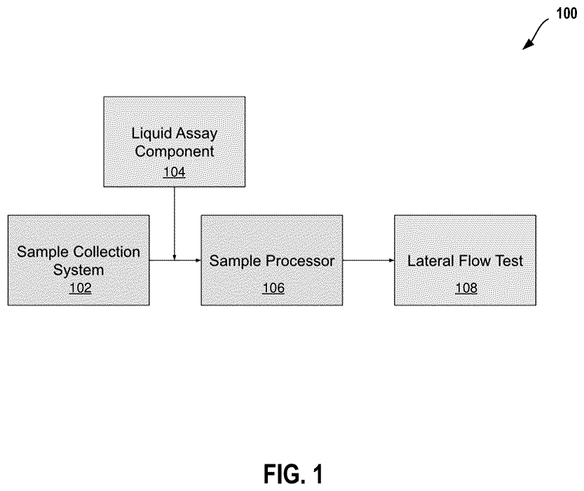

FIG. 1 illustrates a block diagram of a system 100 to detect allergens using a lateral flow test, according to an embodiment. Moreover, FIG. 1 shows the order in which the different components of the device are added in the process/used. Table 1 explains each of these steps in more details.

Table 1 below provides sample details for the system 100 of FIG. 1, according to an embodiment.

| Subcomponent Title | Technical Description |
|---|---|
| Sample Collection | Method of collecting samples into the device. Allows a user to insert a sample/food |
| 102 | into the processor without external tools or potential food contact. |
| Liquid Assay | Liquid containing control lateral flow test liquid (e.g., antigen or antibody), water or |
| 104 | other liquids to dilute the sample, and biologics to suspend allergens (e.g., gluten) |
| | from the food sample. |
| Sample Processor | Reduces the input sample into a consistent paste/liquid such that regardless of initial |
| 106 | condition, it can be run on a lateral flow test. |
| Lateral Flow Test | Using the processed liquid, detects for both the control lateral flow test liquid and the |
| 108 | presence of allergen (e.g., gluten). |

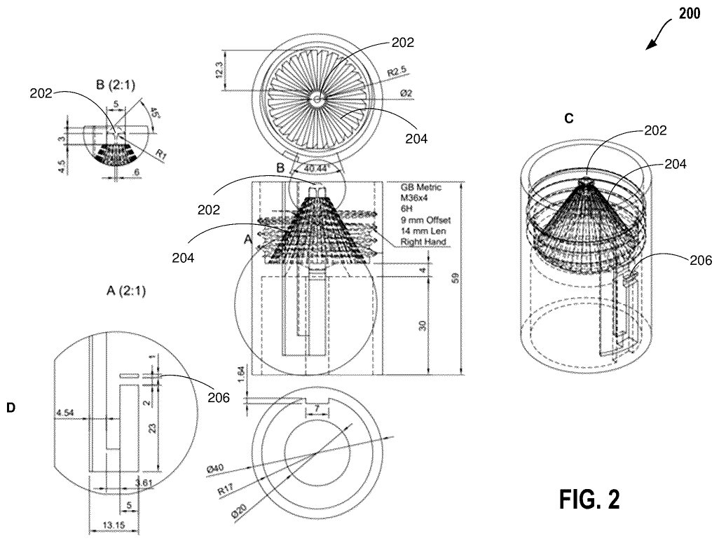

FIG. 2 illustrates various views of a food sample processor 200 that may be used to provide the sample processor of FIG. 1, according to an embodiment.

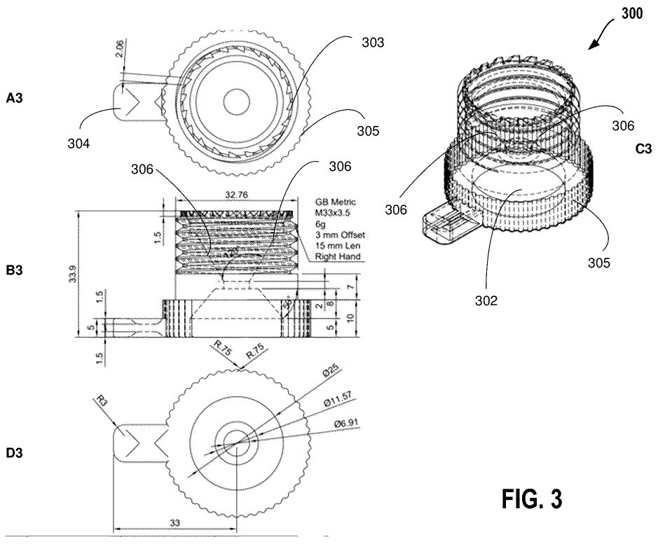

FIG. 2 shows various views of the base of the processor, including a side view A, top view B, a perspective view C, and front view D. In an embodiment, the processor 200 is 3D printed using polylactic acid (PLA) filament. As shown in FIG. 2, the base of the processor includes a cone-shaped grinding portion 204 (which would engage with a matching cone-shaped grinding portion 306 of FIG. 3) with a plurality of ridges in order to better break up the sample. One or more of the ridges may be slanted in one embodiment (e.g., such as shown in item B of FIG. 2). In an alternative embodiment, the ridges may be made using a metallic material such as stainless steel, carbon steel, or other material used to make cutting blades/knives.

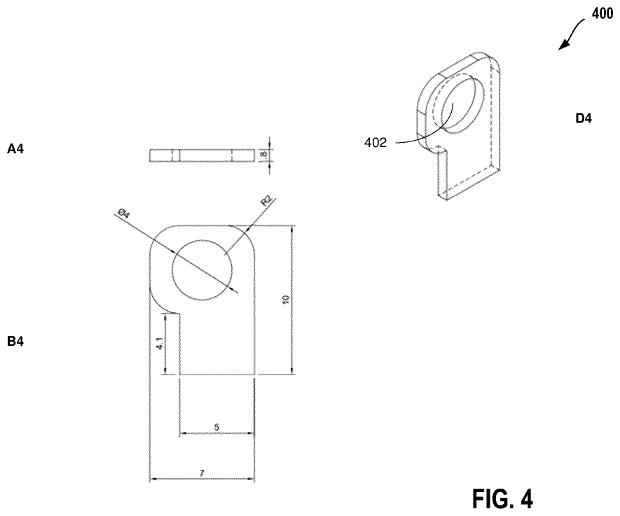

In one embodiment, the tip at the top of the processor grinding portion 204 (Below B(2:1), item 202) is wider at the top and thinner at the bottom (e.g., tapered) to ensure that liquid may escape through the lid and into the processing body when it is punctured. If the tip point is constructed with a simple cone, no liquid may pass from the reservoir 302 of FIG. 3 by, e.g., due to water tension. The liquid collection component A(2:1) has a slot 206 for a pull tab 400 (shown in FIG. 4), and a channel for the liquid to flow into before it reaches the lateral flow test material (e.g., item 504 in FIG. 5). In alternative embodiments, the device may have multiple channels, or wider channels, in order to store multiple lateral flow tests. This would allow detection of multiple allergens at once.

Also, while some figures show sample dimensions in millimeters (mm), embodiments are not limited to such dimensions and smaller or larger dimensions may be present in a device made according to various embodiments.

FIG. 3 illustrates various views of a lid 300, according to an embodiment. The views shown include a bottom view A3, side view B3, perspective view C3, and top view D3. The lid 300 may be used to collect a sample and release a stored (e.g., antibody) liquid from a liquid reservoir 302 for test purposes, according to an embodiment.

FIG. 4 illustrates various views of a pull tab 400, according to an embodiment. The views shown include a side view A4, top view B4, and a perspective view D4. As shown, a hole 402 may be optionally included in the pull tab 400 so that a string can be attached in order to make it easier to pull the tab. Other embodiments may include a larger pull tab, or a pull tab that includes an indentation in order to increase the grip that a user can get on the pull tab. The rest of the body may use a different material such as rubber, e.g., for a more tactile feel and/or better user grip.

FIG. 5 illustrates the various portions of an allergen detection device 500, according to an embodiment. FIG. 5 shows the detection device fully integrated. The liquid is stored in the top portion as discussed with reference to FIG. 3. A test media 504 (e.g., lateral flow test material) is slid into a slot in the bottom portion. The test media and channel 502 may be covered with (e.g., a thin) translucent plastic sheeting in order to allow the user to visually read the test. Additionally, in at least one embodiment, top of the lid may be covered with (e.g., a thin) translucent plastic sheeting in order to allow the user to observe liquid flow from the reservoir 302 of FIG. 3.

Referring to FIGS. 1-5, the top portion of FIG. 5 (previously shown with more views in FIG. 3) can be inserted into the bottom portion of FIG. 5 (previously shown with more views in FIG. 2) with the food being tested between the two portions. A twisting motion of the top portion allows for the release of liquid from reservoir 302 of FIG. 3 to process the food sample being tested. In an embodiment, liquid reservoir 302 stores one or more of: a lateral flow test liquid, water (e.g., distilled, deionized, or otherwise chemically inert water), combinations thereof (e.g., a 50/50 or 75/25 combination (or other combination ratio) of lateral flow test liquid and water). Alternatively, liquid in reservoir 302 may consist of ethanol, or a combination of ethanol and lateral flow test liquid (e.g., with various ratios, including 50/50, 75/25, or another ratio). However, combination of ethanol may require that that operations be performed during a specific time period to avoid the ethanol damaging or disintegrating the lateral flow test liquid.

After sufficient liquid (e.g., where liquid flows to the processor portion) is released by the twisting motion of the top portion against the bottom portion, a pull tab (shown on the side of the bottom portion and in FIG. 4) is used to allow the flow of the generated liquid into a lateral flow test. As discussed herein, “sufficient liquid” refers to an amount of liquid that meets the minimum liquid required by the lateral flow test to function properly (e.g., enough liquid to soak the requisite portion of the lateral flow test medium 504). Moreover, use of a pull tab is envisioned to unblock the path of the liquid towards the lateral flow test. Other blocking mechanisms and/or flow control device(s) may be used separately or in combination with the pull tab, such as one or more of: a valve, a screw, a membrane (that is punctured or removed), etc.

Referring to FIG. 5, a cover 502 may be provided to protect the liquid from outside elements. In an embodiment, the cover 502 is made of a transparent material to allow for observation of the liquid flow. The liquid flows into a lateral flow test material 504, e.g., via the channel shown and covered with (e.g., transparent) cover 502. As shown in FIG. 5, the lateral flow test material 504 may include one or more indicators 506 to visually illustrate whether an allergen is present in the food sample being tested (e.g., by lines or another type of indicator changing colors).

Additionally, while some embodiments include the lateral flow reservoir 302 in the lid 300, the reservoir may be provided in the food processor portion 200 (e.g., around a perimeter of the food processor 200 to allow mixture with the grinded food sample prior to saturating the lateral flow test material). Also, the cone shaped grinder may be inverted such that the cone portion at the bottom portion of the food grinder is indented (i.e., the reverse of what is shown in FIG. 2), while the top portion of the grinder formed by the cone-shaped portion 306 of FIG. 3 is extruded (i.e., reversing the shape of the lid grinder portion 306 in FIG. 3). Alternatively, the grinder portions may include flat surfaces (i.e., no longer concave).

The pull tab 400 may allow for timing to elapse prior to activating the lateral flow test to ensure that the sample has had sufficient time to process with the liquid in reservoir 302, e.g., from a lateral flow testing kit. If the combination does not have time to process, the test will not be able to determine if there is allergen within the sample.

In one embodiment, the opening that holds the pull tab (called a pull tab “slot”) is slanted to ensure the sample does not clog the slot. In another embodiment, the pull tab slot is vertical (i.e., along an axis perpendicular to a rotational plane of the lid 300 and processor 200 engagement, e.g., straight up and down to utilize gravity assist for processed sample flow). Also, the channel design below the pull tab slot (502) may help ensure that the liquid does not overwhelm the test by limiting or restricting the flow of the liquid. For example, if the liquid flowed directly to the bottom of the test, the test could become over-saturated.

Referring to FIGS. 3 and 5, the lid 300 may be printed out of PLA. The lid optionally includes teeth 303 along its edge to better process the food sample. The liquid component may be stored in the lid (in cone 302), which may include a membrane that is punctured (e.g., by the tip 202 of FIG. 2) in order to add the liquid to the food sample. In an embodiment, the membrane is made from hot glue (e.g., ethylene vinyl acetate) or hot melt adhesive, etc., but other material may be used such as silicon, rubber, plastic, glass, etc. A handle 304 may be included on the lid to allow users to scoop liquid samples and/or make it easier to tighten the lid by providing more leverage. The lid may also have ridges 305 on the outside to make it easier to grip.

In another embodiment, the lid may have multiple reservoirs (instead of the single reservoir 302 shown in FIG. 3) for storage of different liquids. Such an approach allows the same device to be utilized for testing different allergens. A selector (not shown) such as a rotary dial on the lid may be used to select which liquid to dispense. In turn, the lateral flow test material 504 of FIG. 5 may be switched depending on the type of allergen being tested.

Alternatively, a separate channel may be provided in the processor 200 of FIG. 2 for a different lateral flow test material to allow the flow of the liquid to a given test material. In this fashion, the same food sample can be tested for more than one allergen.

In another embodiment, different liquids may be combined (e.g., a short period of time, such as approximately 5 to 15-30 minutes) to allow for testing of multiple allergens in the same food sample at the same time. This latter approach could save on costs and weight of the allergen detection device.

In one embodiment, the apparatus of FIG. 5 has a cylindrical shape (such as shown in FIG. 5 (but embodiments are not limited to a cylindrical shape and the detection device may have other shapes, such as square, flat cube, cubical, etc.). The size of the apparatus may be sufficiently small to make the apparatus easy to carry in a bag, pocket, etc. For example, the allergen detection apparatus may be approximately 4 inches by 2 inches, or less. Alternative embodiments could include a lid and/or sample processor having a rectangular shape, e.g., to make it easier to grip/turn, for example, by providing more physical leverage through the rectangular sides. Other embodiments may include a spring-loaded component to assist in grinding the food, or an electronic component that grinds the food for users. This in turn will increase usability for users who may have a variety of disabilities.

Furthermore, the spring-loaded implementation may operate by a user rotating the lid against the sample processor portion to load a spring. Each rotation is envisioned to be easier than directly grinding the food, for example, by using a gear ratio that makes the rotation easier for a disabled user. A user may then release the loaded/wound spring by pressing a button. For the electronic grinder, a motor, a battery, and a control switch/button may be provided in the top and/or bottom portion of the apparatus of FIG. 5. Upon pressing the control switch, a user may cause the battery to operate the motor to effect rotation of the sample processor grinder portion.

Accordingly, features in one or more embodiments include:
(1) grinding component with multiple ridges/blades to ensure consistent grinding (see, e.g., FIGS. 3 and/or 5);
(2) handle on the griding component, e.g., to provide more physical leverage to operate the griding component (see, e.g., FIGS. 3 and/or 5);
(3) no electronics involved (in contrast to other testing devices on the market), which removes the potential for issues and makes the test more simple and robust;
(4) self contained, i.e., users do not need to buy capsules separately or use a separate food processor;
(5) the pull tab 400 of FIG. 4 allows the food sample to sit/combine with the testing liquid, producing a better and more accurate result;
(6) the slot for the test material 504 that allows the liquid to flow from the bottom of the device, e.g., avoiding flooding of the test media versus if the liquid is just added at the top of the test material; and/or
(7) a single-use device, e.g., to avoid cross-contamination by a different food sample.

The following examples pertain to further embodiments. Example 1 includes an apparatus comprising: a food processor to receive a food sample; and a lid to engage the food processor mechanically to grind the food sample; wherein a mixture of a lateral flow test liquid and the ground food sample is to saturate a portion of a lateral flow test material to indicate whether the food sample includes an allergen.

Example 2 includes the apparatus of example 1, wherein the lid comprises the lateral flow test liquid. Example 3 includes the apparatus of example 1, wherein the food processor includes the lateral flow test material. Example 4 includes the apparatus of example 1, wherein the food processor includes a cone-shaped grinding portion to engage the lid mechanically to grind the food sample. Example 5 includes the apparatus of example 4, wherein the cone-shaped grinding portion of the food processor comprises a plurality of ridges.

Example 6 includes the apparatus of example 5, wherein one or more of the plurality of ridges are slanted. Example 7 includes the apparatus of example 4, wherein a tip of the cone-shaped grinding portion of the food processor is tapered. Example 8 includes the apparatus of example 1, wherein the lateral flow test liquid is to be mixed with water, wherein the water includes one or more of: distilled water, deionized water, chemically inert water, or combinations thereof. Example 9 includes the apparatus of example 1, wherein the apparatus is a single-use device.

Example 10 includes the apparatus of example 1, wherein the lateral flow test liquid is to be mixed with ethanol. Example 11 includes the apparatus of example 1, wherein the lid comprises at least one handle. Example 12 includes the apparatus of example 1, wherein the lateral flow test liquid includes at least one antibody. Example 13 includes the apparatus of example 1, wherein the lateral flow test material includes one or more indicators to visually illustrate whether the allergen is present in the food sample. Example 14 includes the apparatus of example 1, further comprising a flow control device to control a rate of the mixture of the lateral flow test liquid and the ground food sample.

Example 15 includes the apparatus of example 14, wherein the flow control device comprises a pull tab, wherein the pull tab includes a hole to receive a string to assist in pulling the pull tab. Example 16 includes the apparatus of example 14, wherein the food processor includes a slot to receive the flow control device. Example 17 includes the apparatus of example 16, wherein the slot is slanted. Example 18 includes the apparatus of example 16, wherein the slot is vertical along an axis perpendicular to a rotational plane of the lid and the processor mechanical engagement. Example 19 includes the apparatus of example 1, further comprising a transparent cover to allow for observation of liquid flow passed or through the flow control device. Example 20 includes the apparatus of example 1, further comprising: (a) one or more lateral flow test liquid reservoirs to each store one or more lateral flow test liquids; and (b) a plurality of lateral flow test materials to test for a plurality of allergens.

Example 21 includes an apparatus comprising means to perform a method as set forth in any preceding example.

References to “one example,” “an example,” etc., indicate that the example described may include a particular feature, structure, or characteristic, but every example may not necessarily include the particular feature, structure, or characteristic. Moreover, such phrases are not necessarily referring to the same example. Further, when a particular feature, structure, or characteristic is described in connection with an example, it is submitted that it is within the knowledge of one skilled in the art to affect such feature, structure, or characteristic in connection with other examples whether or not explicitly described.

Moreover, in the various examples described above, unless specifically noted otherwise, disjunctive language such as the phrase “at least one of A, B, or C” or “A, B, and/or C” is intended to be understood to mean either A, B, or C, or any combination thereof (i.e. A and B, A and C, B and C, and A, B and C).

The specification and drawings are, accordingly, to be regarded in an illustrative rather than a restrictive sense. It will, however, be evident that various modifications and changes may be made thereunto without departing from the broader spirit and scope of the disclosure as set forth in the claims.

In the following description, numerous specific details are set forth to provide a more thorough understanding. However, it will be apparent to one of skill in the art that the embodiments described herein may be practiced without one or more of these specific details. In other instances, well-known features have not been described to avoid obscuring the details of the present embodiments.

Reference in the specification to “one embodiment” or “an embodiment” means that a particular feature, structure, and/or characteristic described in connection with the embodiment may be included in at least an implementation. The appearances of the phrase “in one embodiment” in various places in the specification may or may not be all referring to the same embodiment.

Also, in the description and claims, the terms “coupled” and “connected,” along with their derivatives, may be used. In some embodiments, “connected” may be used to indicate that two or more elements are in direct physical contact with each other. “Coupled” may mean that two or more elements are in direct physical or electrical contact. However, “coupled” may also mean that two or more elements may not be in direct contact with each other, but may still cooperate or interact with each other.

Thus, although embodiments have been described in language specific to structural features and/or methodological acts, it is to be understood that claimed subject matter may not be limited to the specific features or acts described. Rather, the specific features and acts are disclosed as sample forms of implementing the claimed subject matter.

1. An apparatus comprising:
a food processor to receive a food sample; and
a lid to engage the food processor mechanically to grind the food sample;
wherein a mixture of a lateral flow test liquid and the ground food sample is to saturate a portion of a lateral flow test material to indicate whether the food sample includes an allergen.

2. The apparatus of claim 1, wherein the lid comprises the lateral flow test liquid.

3. The apparatus of claim 1, wherein the food processor includes the lateral flow test material.

4. The apparatus of claim 1, wherein the food processor includes a cone-shaped grinding portion to engage the lid mechanically to grind the food sample.

5. The apparatus of claim 4, wherein the cone-shaped grinding portion of the food processor comprises a plurality of ridges.

6. The apparatus of claim 5, wherein one or more of the plurality of ridges are slanted.

7. The apparatus of claim 4, wherein a tip of the cone-shaped grinding portion of the food processor is tapered.

8. The apparatus of claim 1, wherein the lateral flow test liquid is to be mixed with water, wherein the water includes one or more of: distilled water, deionized water, chemically inert water, or combinations thereof.

9. The apparatus of claim 1, wherein the apparatus is a single-use device.

10. The apparatus of claim 1, wherein the lateral flow test liquid is to be mixed with ethanol.

11. The apparatus of claim 1, wherein the lid comprises at least one handle.

12. The apparatus of claim 1, wherein the lateral flow test liquid includes at least one antibody.

13. The apparatus of claim 1, wherein the lateral flow test material includes one or more indicators to visually illustrate whether the allergen is present in the food sample.

14. The apparatus of claim 1, further comprising a flow control device to control a rate of the mixture of the lateral flow test liquid and the ground food sample.

15. The apparatus of claim 14, wherein the flow control device comprises a pull tab, wherein the pull tab includes a hole to receive a string to assist in pulling the pull tab.

16. The apparatus of claim 14, wherein the food processor includes a slot to receive the flow control device.

17. The apparatus of claim 16, wherein the slot is slanted.

18. The apparatus of claim 16, wherein the slot is vertical along an axis perpendicular to a rotational plane of the lid and the processor mechanical engagement.

19. The apparatus of claim 1, further comprising a transparent cover to allow for observation of liquid flow passed or through the flow control device.

20. The apparatus of claim 1, further comprising:
(a) one or more lateral flow test liquid reservoirs to each store one or more lateral flow test liquids; and
(b) a plurality of lateral flow test materials to test for a plurality of allergens.
